(19) United States
(12) Patent Application Publication
(10) Pub. No.: US 2021/0082567 A1
(43) Pub. Date: Mar. 18, 2021

(54) METHOD FOR SUPPORTING VIEWING OF IMAGES AND APPARATUS USING SAME

(71) Applicant: VUNO, INC., Seoul (KR)

(72) Inventor: Seungho LEE, Gyeonggi-do (KR)

(21) Appl. No.: 16/963,700

(22) PCT Filed: Dec. 7, 2018
(86) PCT No.: PCT/KR2018/015483

§ 371 Date: Jul. 21, 2020

(30) Foreign Application Priority Data
Jan. 22, 2018 (KR) 10-2018-0007526

(51) Int. Cl. G16H 30/40; G06F 3/0485; G06T 7/00; G16H 50/20

(52) U.S. Cl. CPC G16H 30/40; G06F 3/0485; G06F 3/0489; G16H 50/20; G06T 7/0012

(57) ABSTRACT

The present invention relates to a method for supporting viewing of images and an apparatus using same. In particular, according to the method of the present invention, the computing device enables sequential viewing of a series of individual images, in response to a specific input of an input device, wherein a switching speed from a first individual image, which is an individual image provided in a current viewing, to a second individual image, which is an individual image provided in the next viewing, is variably increased or decreased according to importance given to at least one of the first individual image and the second individual image.

### TECHNICAL FIELD

Example embodiments relate to a method of supporting viewing of images and an apparatus using the same, and more particularly, to a method in which a computing apparatus enables sequential viewing of a series of individual images in response to a specific input of an input device, and a switching speed from a first individual image that is an individual image provided in a current viewing to a second individual image that is an individual image provided in a subsequent viewing variably increases or decreases according to an importance associated with at least one of the first individual image and the second individual image.

### RELATED ART

Various methods are employed to enable viewing of a plurality of associated images at a high speed. For example, in the case of a plurality of associated slice images, such as, a computed tomography (CT) image, a user, for example, a doctor, generally verifies presence or absence of each lesion and a state thereof by quickly turning an individual slice image to a slice image adjacent thereto through a manipulation of an input device.

For example, medical images, such as chest CT images, which are widely used for analyzing lesions for diagnostic use, are frequently used for reading because abnormalities inside the body, for example, lungs, bronchus, and heart, may be observed. Such reading of chest CT images is performed by examining a series of individual slice images from a lowermost or uppermost part of a photographing area according to three-dimensional (3D) characteristics of a tomographic image. In general, as a switching speed between images performed using a mouse drag, a wheel rotation, etc., increases, an amount of time required for the entire reading decreases, but it is difficult to read the image closely. Also, since chest CT images usually do not include only the lungs, it takes time to switch between images that are not necessary for actual reading.

Some of the findings readable through chest CT images may be easily overlooked by human doctors since they are not easy to read so that radiologists and doctors may distinguish corresponding features and shapes only after many years of training. If a difficulty of corresponding reading is high, such as pulmonary nodule, it may be overlooked even if a doctor pays great attention.

In order to assist in the reading of images that may be easily overlooked by humans, a need for computer aided diagnosis (CAD) has emerged in the art. However, the conventional CAD technology simply assists a doctor in such reading in a very limited area. Alternatively, although the conventional CAD technology supports a lesion diagnosis to some extent, no special convenience is provided for a doctor to read the entire image in terms of a user interface. For example, an apparatus and method for assisting a lesion diagnosis using a combination of a conventional machine learning algorithm and a deep learning algorithm is disclosed in Korean Patent Laid-Open Publication No. 10-2017-0047423.

As described above, reading of lesions using a computer-assisted diagnosis (CAD) may be performed through a process of initially, specifying an area suspected as a lesion and evaluating an importance (e.g., confidence, malignity, etc.) of the area. For example, if a plurality of lesions (e.g., nodules) are found in the lung, only lesion areas where the malignity is expected to be high may need to be investigated closely, and areas where only lesions with relatively low malignity are found or where any lesion is not found may pass quickly.

However, since a plurality of lesions are present, which of the lesions is really serious is unknown before the reading. The same extent of efforts for reading is required even with respect to a lesion of which actual malignity is not high or a lesion that is not expected to be malignant, which may lead to degrading efficiency in many cases.

Accordingly, to solve the above issues, proposed are herein a method of improving efficiency in reading the entire image by providing a convenience of, even with respect to a conventional lesion detection system, flexibly adjusting an image switching speed between an area where a suspected lesion is present and an area where the suspected lesion is absent and significantly decreasing the image switching speed with respect to an image corresponding to locations at which lesions having a high importance (e.g., confidence, malignity, etc.) are present among the detected lesions, and an apparatus using the same.

### PRIOR ART

Patent Document 1: KR10-2017-0047423 A

### DETAILED DESCRIPTION

### Technical Subject

Example embodiments are to improve a viewing efficiency by allowing further close viewing (particularly, reading) to be performed on an area with a highest importance in an image (particularly, a medical image) and by allowing fast switching between an image of a position corresponding to an area with a relatively low importance or no issue (e.g., a lesion) and a subsequent image.

In detail, example embodiments are to focus on a lesion that is required to be substantially viewed by providing a user interface capable of adjusting a switching speed between images based on a computer-calculated importance.

Example embodiments are to improve an efficiency in viewing images such that a user may verify a larger number of images within a relatively short period of time, and to improve an analysis accuracy by, particularly, assisting a reader to derive accurate diagnostic results from medical images.

### Solution

Characteristic constitutions of the disclosure to accomplish the aforementioned objectives and to achieve characteristic effects of the disclosure are as follows:

According to an aspect of example embodiments, there is provided a method of supporting viewing of images, wherein a computing apparatus enables sequential viewing of a series of individual images in response to a specific input of an input device, and a switching speed from a first individual image that is an individual image provided in a current viewing to a second individual image that is an individual image provided in a subsequent viewing variably increases or decreases according to an importance associated with at least one of the first individual image and the second individual image.

According to another aspect of example embodiments, there is provided a computer program stored in a non-transitory computer-readable storage medium including instructions that cause a computing apparatus to perform the image viewing supporting method.

According to still another aspect of example embodiments, there is provided a computing apparatus for supporting viewing of images, the apparatus including: a communicator configured to acquire a specific input of an input device; and a processor configured to enable sequential viewing of individual images in response to the specific input, wherein the processor is configured to enable a switching speed from a first individual image that is an individual image provided in a current viewing to a second individual image that is an individual image provided in a subsequent viewing to variably increase or decrease according to an importance associated with at least one of the first individual image and the second individual image.

### Effects

According to some example embodiments, since it is possible to adjust an image switching speed based on an importance or according to a manipulation of a user without compromising the existing image viewing method, the user may concentrate on analysis of areas of an image to be significantly viewed, which may lead to improving an image viewing (reading) efficiency.

According to some example embodiments, since a viewing efficiency is improved, a doctor may derive further accurate diagnostic results within a relatively short period of time in a medical portion, which may lead to improving a speed and quality of reading and to innovating a workflow in a medical field.

According to some example embodiments, it may apply to various images, particularly, medical images used in hospitals in the related art. For example, the example embodiments may apply as is to systems, such as, for example, three-dimensionally acquired ultrasound images, magnetic resonance imaging (MRI) images, etc. Therefore, a method proposed herein is not dependent on a particular type of an image or platform.

### BRIEF DESCRIPTION OF DRAWINGS

Example embodiments will be described in more in detail with reference to the following figures that are simply a portion of the example embodiments and those skilled in the art to which this disclosure pertains may readily acquire other figures based on the figures without an inventive work being made:

FIG. 1 is a diagram illustrating an example of a configuration of a computing apparatus configured to perform a method (hereinafter, also referred to as an “image viewing supporting method”) of supporting viewing of images according to an example embodiment;

FIG. 2 is a diagram illustrating an example of hardware or software components of a computing apparatus configured to perform an image viewing supporting method according to an example embodiment;

FIG. 3 is a flowchart illustrating an example of an image viewing supporting method according to an example embodiment;

FIG. 4 illustrates an example of describing an image viewing supporting method according to an example embodiment;

FIG. 5 illustrates an example of describing switching between images according to the example embodiment of FIG. 4;

FIG. 6 illustrates another example of describing an image viewing supporting method according to an example embodiment; and

FIG. 7 illustrates an example of describing switching between images according to the example embodiment of FIG. 6.

### BEST MODE

The following detailed description of this disclosure is described with reference to the accompanying drawings in which specific example embodiments of the disclosure are illustrated as examples, to fully describe purposes, technical solutions, and advantages of the disclosure. The example embodiments are described in detail enough for those skilled in the art to carry out the disclosure.

The terms “image” and “image data” used throughout the detailed description and the claims herein refer to multi-dimensional data that includes discrete image factors (e.g., a pixel in a two-dimensional (2D) image and a voxel in a three-dimensional (3D) image).

For example, the term “image” may refer to a medical image of a subject collected by cone-beam computed tomography (CT), magnetic resonance imaging (MRI), an ultrasound system, or known other medical imaging systems in the related art. Also, the image may be provided in a non-medical context, for example, a remote sensing system, and an electron microscopy, and the like.

The term “image” used throughout the detailed description and the claims may refer to an image visible with an eye (e.g., displayed on a video screen) or a digital representation of an image (e.g., a file corresponding to a pixel output of a CT, an MRI detector, and the like).

For clarity of description, although cone-beam CT (CBCT) image data is illustrated in the drawings as image modality, it will be apparent to those skilled in the art that image forms used in various example embodiments include X-ray images, MRI, CT, positron emission tomography (PET), PET-CT, single photo emission computed tomography (SPECT), SPECT-CT, MR-PET, 3D ultra sound images, etc., without being limited thereto.

The term “Digital Imaging and Communications in Medicine (DICOM)” standard used throughout the detailed description and the claims is a generic term for a plurality of standards used for digital image representation and communication in medical devices. The DICOM standard is published by the American College of Radiology (ACR) and the National Electrical Manufacturers Association (NEMA).

Also, the term “Picture Archiving and Communication System (PACS)” used throughout the detailed description and the claims is a term for systems that perform storage, processing, and transmission according to the DICOM standard. A medical image acquired using digital medical imaging equipment such as X-ray, CT, and MRI may be stored in a DICOM format and may be transmitted to a terminal inside or outside a hospital over a network. Here, a reading result and a medical record may be added to the medical image.

Further, the term “training” or “learning” used throughout the detailed description and the claims refers to performing a machine learning through computing according to a procedure and it will be apparent to those skilled in the art that the term is not intended to refer to a mental action such as an educational activity of a human.

Also, the terms “comprises/includes” used throughout the detailed description and the claims and modifications thereof are not intended to exclude other technical features, additions, components, or operations. Also, “single” or “one” is used to indicate at least one and “another” is limited to at least second or more.

Those skilled in the art may clearly understand a portion of other purposes, advantages, and features of the disclosure from this specification and another portion thereof from implementations of the disclosure. The following examples and drawings are provided as examples only and not to limit the disclosure. Therefore, the detailed description disclosed herein should not be interpreted as a limiting meaning with respect to a specific structure or function and should be interpreted as representative basic data that provides guidelines such that those skilled in the art may variously implement the disclosure as substantially suitable detailed structures.

Further, the disclosure may include any possible combinations of example embodiments described herein. It should be understood that, although various example embodiments differ from each other, they do not need to be exclusive. For example, a specific shape, structure, and feature described herein may be implemented as another example embodiment without departing from the spirit and scope of the disclosure. Also, it should be understood that a position or an arrangement of an individual component of each disclosed example embodiment may be modified without departing from the spirit and scope of the disclosure. Accordingly, the following detailed description is not to be construed as being limiting and the scope of the disclosure, if properly described, is limited by the claims, their equivalents, and all variations within the scope of the claims. In the drawings, like reference numerals refer to like elements throughout.

Unless the context clearly indicates otherwise, the singular forms “a,” “an,” and “the,” are intended to include the plural forms as well. Also, when description related to a known configuration or function is deemed to render the present disclosure ambiguous, the corresponding description is omitted.

Hereinafter, example embodiments of the disclosure are described in detail with reference to the accompanying drawings such that those skilled in the art may easily perform the example embodiments.

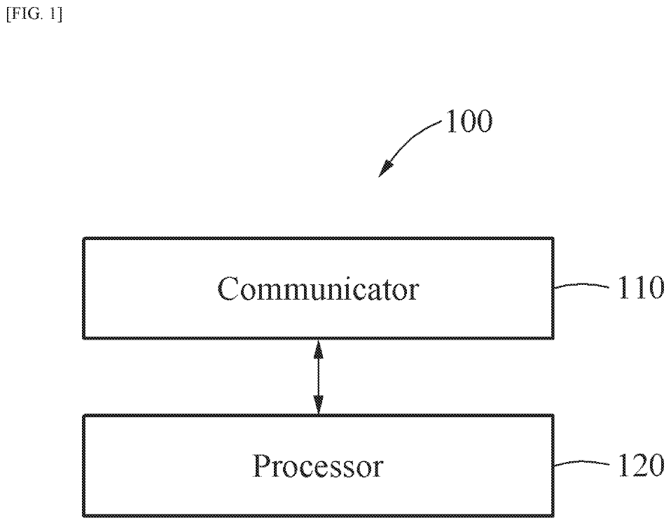

FIG. 1 is a diagram illustrating an example of a configuration of a computing apparatus configured to perform an image viewing supporting method according to an example embodiment.

Referring to FIG. 1, a computing apparatus 100 according to an example embodiment includes a communicator 110 and a processor 120, and may directly or indirectly communicate with an external computing apparatus (not shown) through the communicator 110.

In detail, the computing apparatus 100 may achieve a desired system performance using a combination of typical computer hardware (e.g., an apparatus including a computer processor, a memory, a storage, an input device and an output device, components of other existing computing apparatuses, etc.; an electronic communication apparatus such as a router, a switch, etc.; an electronic information storage system such as a network-attached storage (NAS) and a storage area network (SAN)) and computer software (i.e., instructions that enable a computing apparatus to function in a specific manner).

The communicator 110 of the computing apparatus 100 may transmit and receive a request and a response with another interacting computing apparatus. As an example, the request and the response may be implemented using the same transmission control protocol (TCP) session. However, it is provided as an example only. For example, the request and the response may be transmitted and received as a user datagram protocol (UDP) datagram. In addition, in a broad sense, the communicator 110 may include a keyboard, a mouse, and other external input devices to receive a command or an instruction, etc., and a printer, a display, and other external input devices.

Also, the processor 120 of the computing apparatus 100 may include a hardware configuration, such as a micro processing unit (MPU), a central processing unit (CPU), a graphics processing unit (GPU), a tensor processing unit (TPU), a cache memory, a data bus, and the like. Also, the processor 120 may further include a software configuration of an application that performs a specific objective, an operating system (OS), and the like.

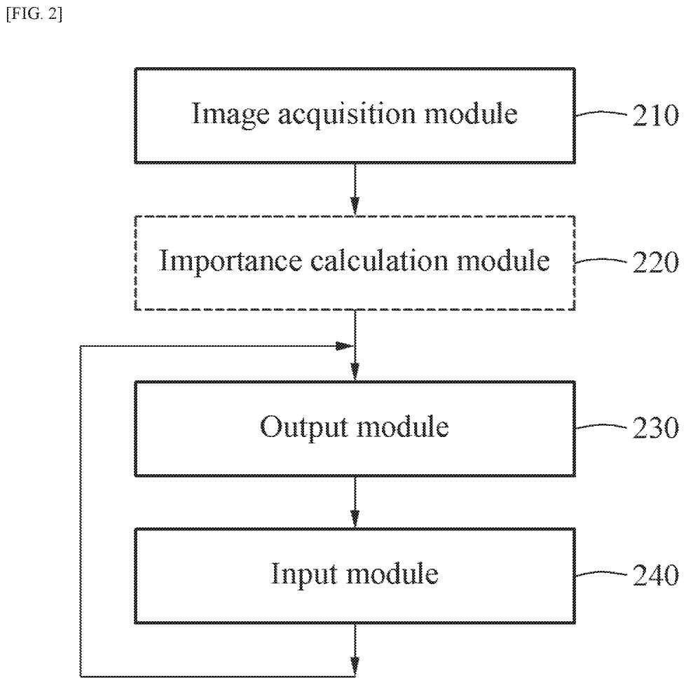

FIG. 2 is a diagram illustrating an example of hardware or software components of a computing apparatus configured to perform an image viewing supporting method according to an example embodiment.

Describing a method and a configuration of an apparatus according to an example embodiment with reference to FIG. 2, the computing apparatus 100 may include an image acquisition module 210 as a component. The image acquisition module 210 is configured to acquire a series of individual images to which the method according to an example embodiment applies. It will be apparent to those skilled in the art that individual modules of FIG. 2 may be configured through, for example, the communicator 110 or the processor 120 included in the computing apparatus 100, or through interaction between the communicator 110 and the processor 120. The series of individual images may be acquired from an external image storage system, such as, for example, an imaging device interacting through the communicator 110 or Picture Archiving and Communication System (PACS). However, it is provided as an example only. For example, the series of individual images may be captured by a (medical) imaging device and transmitted to the PACS according to the DICOM standard and then, acquired by the image acquisition module 210 of the computing apparatus 100. When the series of individual images are acquired as a medical image, the series of individual images may be continuous due to their characteristics. That is, a change between adjacent individual images may not be discontinuous.

The acquired individual images may be forwarded to an importance calculation module 220. The importance calculation module 220 may calculate an importance of each of the individual images. Alternatively, when acquiring the individual images, the importance of each of the individual images may also be acquired.

If an individual image is a medical image, an importance may be scores indicating, for example, a confidence that a suspected lesion detected in the individual image is an actual lesion, a malignity of the suspected lesion, and the like, or a value that is calculated based on importance factors including at least one of the confidence and the malignity. In the case of the medical image, the importance calculation module 220 may be a lesion determination model configured to determine a lesion or a module associated therewith. The importance is intended to alert a reader to the corresponding suspected lesion.

An example of the importance calculation module or the lesion determination model 220 may include a deep learning model, which is in a structure in which an artificial neural network is stacked in multiple layers. That is, it may be represented as a deep neural network as a meaning of a deep structured network. The network may be trained by learning a large amount of data in a multilayered network structure and thereby automatically learning features of each image and accordingly, minimizing an error of an objective function, that is, an error of a lesion determination accuracy. It is compared to connectivity between neural cells of the human brain. Such a deep neural network is becoming a next generation model of artificial intelligence (AI). In particular, among examples of the deep learning model, a convolutional neural network (CNN) may be a model suitable for classifying images and may extract various levels of features ranging from a low level feature, such as a dot, a line, a surface, etc., to a high level feature, which is complex and significant, by repeating a convolution layer for generating a feature map of each area using a plurality of filters and a sub-sampling layer for extracting an invariant feature against a change in a position or a rotation through a reduction in a size of the feature map. In the case of using a finally extracted feature as an input value of an existing determination model, a determination model with a higher accuracy may be constructed.

However, it will be understood by those skilled in the art that the importance calculation module or the lesion determination model is not limited to the CNN. Accordingly, various types of machine learning models or statistical models may be used.

When the importance of the individual image is calculated by the importance calculation module 220, the individual image may be forwarded to an output module 230. The output module 230 may provide the individual image to an external entity through a user interface displayed on, for example, a predetermined output device. Here, additional information of the individual information may also be provided.

Here, the external entity may include a user of the computing apparatus 100, a manager, a medical expert in charge of the subject, and, in addition thereto, may also include any types of entities that require the individual image and additional information (read assist information, lesion items, and the like).

Also, an input module 240 may switch a current viewing image displayed on the output module 230 or adjust a switching speed thereof in response to a specific input or a predetermined manipulation.

Functions and effects of components shown in FIG. 2 are further described below. Although the components of FIG. 2 are illustrated in a single computing apparatus for clarity of description, the computing apparatus 100 that performs the method of the disclosure may be configured such that a plurality of apparatuses may interact with each other.

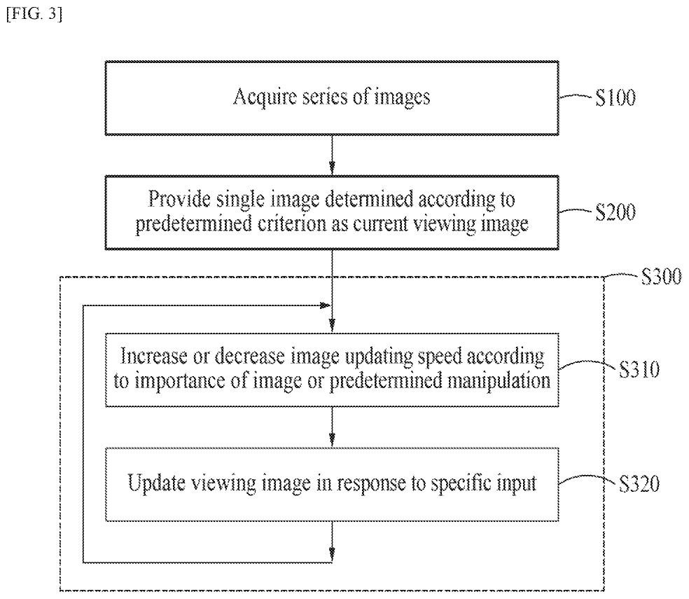

Hereinafter, an image viewing supporting method according to an example embodiment is further described with reference to FIGS. 3 to 7.

FIG. 3 is a flowchart illustrating an example of an image viewing supporting method according to an example embodiment.

Referring to FIG. 3, the image viewing supporting method overall refers to a method in which a computing apparatus enables sequential viewing of a series of individual images in response to a specific input of an input device. Here, a switching speed from a first individual image that is an individual image provided in a current viewing to a second individual image that is an individual image provided in a subsequent viewing variably increases or decreases according to an importance associated with at least one of the first individual image and the second individual image. Here, the specific input may be a manipulation generally used to switch between images, such as for example, a wheel rotation of a mouse, a drag of a mouse or a touchpad, an input of pressing an arrow key on a keyboard, and the like. The specific input may be iteratively input, which may be measured based on an input amount. For example, the wheel rotation of the mouse may be designed to perform an intended action if an input amount iteratively accumulated reaches a desired value. Herein, the intended action may be, for example, an action of switching between images.

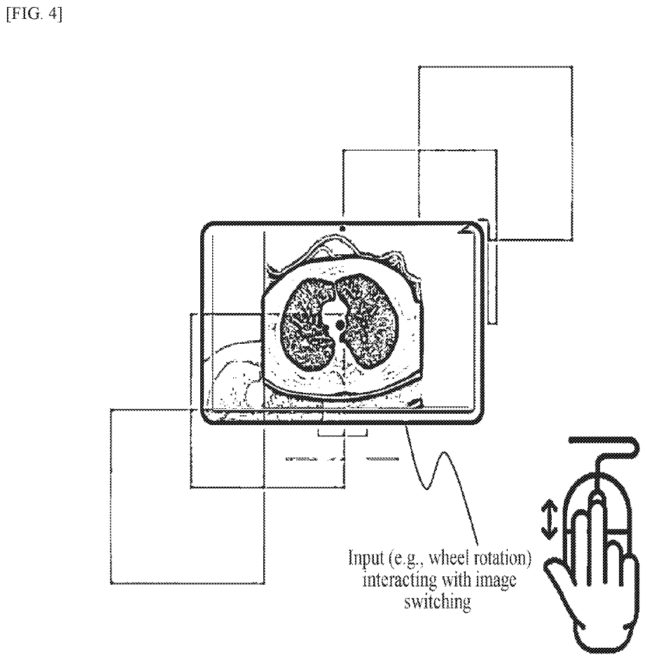

In detail, referring to FIG. 3, the image viewing supporting method may include operation S100 of acquiring, by the image acquisition module 210 implemented by the computing apparatus 100, or supporting another apparatus (not shown) interacting through the communicator 110 to acquire the series of individual images. Such an image may be, for example, an axial image of chest CT displayed through a user interface of FIG. 4.

Although a process of reading a lung related lesion, such as nodule, is illustrated for convenience of illustration, it is provided as an example only. The present disclosure may apply to various types of images.

The image viewing supporting method further includes operation S200 of providing, by the output module 230 implemented by the computing apparatus 100, supporting providing of a single image determined according to a predetermined criterion among the series of individual images as a current viewing image.

For example, the predetermined criterion may be a criterion used to select an individual image having an earliest serial number or a latest serial number among the series of individual images as the single image.

Referring again to FIG. 3, the image viewing supporting method further includes operation S300 of repeatedly updating, by the computing apparatus 100, an image provided as the current viewing image with an individual image determined to be provided in a subsequent viewing based on a directivity corresponding to the specific input. In operation S300, a speed of the updating increases or decreases based on an importance of the current viewing image and at least one image adjacent to the current viewing image or according to a predetermined manipulation.

Here, the directivity corresponding to the specific input refers to a standard for determining whether the specific input is to switch a current viewing image to a previous image or to switch the current viewing image to a subsequent image. For example, a user may press a left arrow key to switch to the previous image and may press a right arrow key to switch to the subsequent image, which is associated with a direction of reading intended by the specific input. Here, a relationship between the “current” viewing image and the “previous” image and the “subsequent” image may be determined based on, for example, a serial number of an image, a sequence in which a corresponding image is captured, and the like.

In detail, operation S300 may include operation S310 of calculating or supporting calculating of a predetermined threshold of an input amount accumulated in response to the specific input; and operation S320 of updating the image provided as the current viewing image with the individual image determined to be provided in the subsequent viewing based on a directivity of the input amount if the input amount accumulated in response to the specific input exceeds the predetermined threshold. Here, the threshold is used to adjust the switching speed between images.

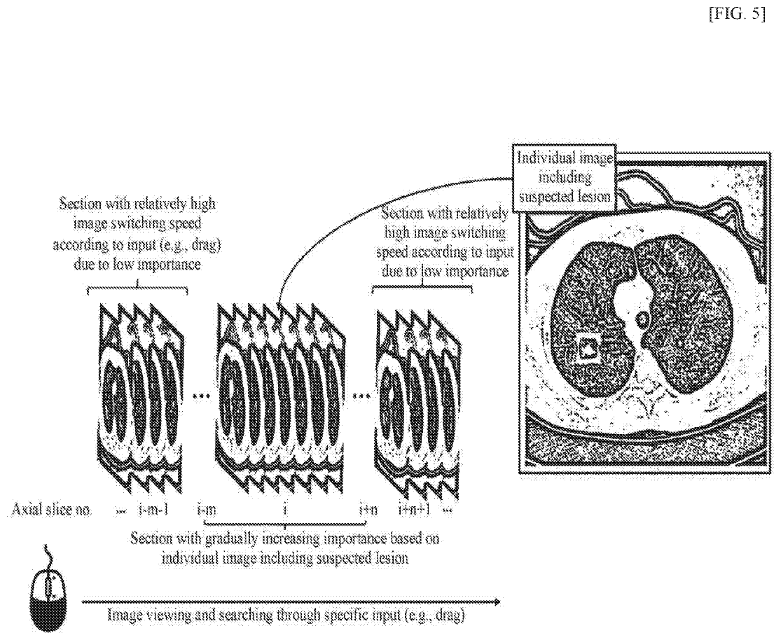

FIG. 4 illustrates an example of describing an image viewing supporting method according to an example embodiment, and FIG. 5 illustrates an example of describing switching between images according to the example embodiment of FIG. 4.

Referring to the example of FIGS. 4 and 5, the threshold is a predetermined value having a function relationship with respect to an importance associated with at least one of the current viewing image, m previous images of the current viewing image, and n subsequent images of the current viewing image or designated according to a predetermined manipulation. Here, m and n≥1 and m and n denote a natural number. The function relationship may be a non-decreasing function to enable switching between images only with a large input amount according to an increase in the importance. For example, when a mouse wheel has a greater rotational angle and a drag has a longer travel distance, switching between images is enabled.

Accordingly, an importance of an image including a suspected lesion may be calculated to be relatively higher compared to those of adjacent images. Therefore, an image switching speed may decrease around the suspected lesion. A plurality of images based on an individual image (see an image with a serial number i of FIG. 5) including the suspected lesion may be displayed for a user for a longer period of time compared to a case to which the present disclosure is not applied.

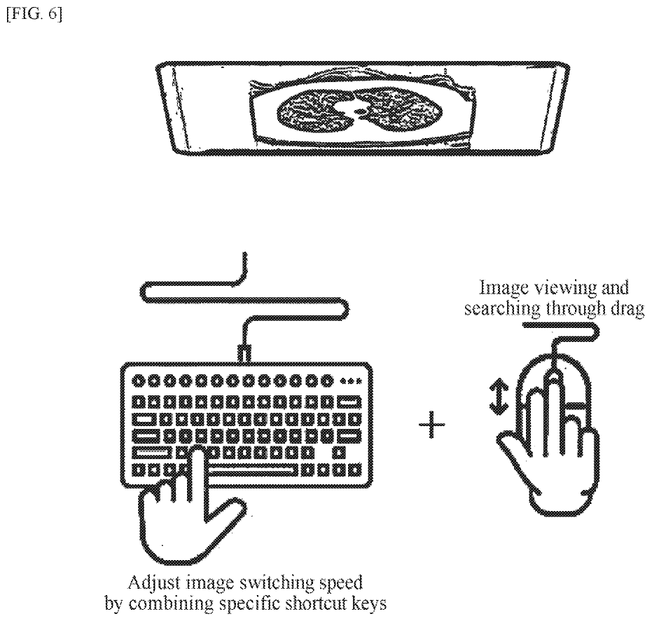

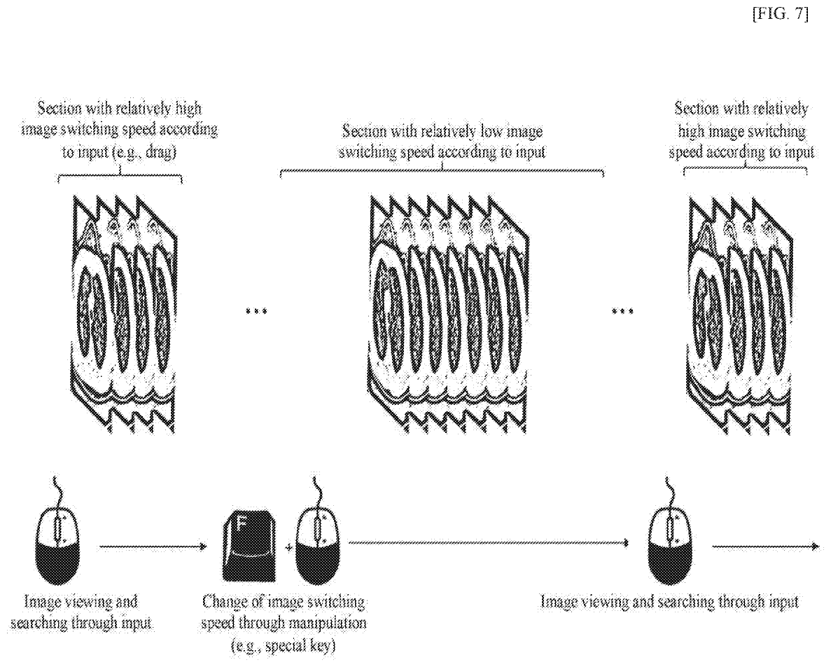

FIG. 6 illustrates another example of describing an image viewing supporting method according to an example embodiment, and FIG. 7 illustrates an example of describing switching between images according to the example embodiment of FIG. 6.

Referring to the other example of FIGS. 6 and 7, the threshold may be value that may be designated or increase or decrease according to a predetermined manipulation. For example, the predetermined manipulation may be a manipulation of pressing a shortcut key. In FIG. 6, a key “F” is pressed as an example. In a state in which a shortcut key is pressed, the threshold may increase. In a state in which the shortcut key is not pressed, the threshold may decrease to an original value.

Accordingly, when a user, for example, a reader, desires to perform more intensive or close viewing, for example, reading, the user may adjust an image switching speed more conveniently according to the predetermined manipulation compared to a case to which the present disclosure is not applied.

As described above with the example embodiments and modifications, according to example embodiments, a user (e.g., a reader) may conveniently and quickly view an image based on an importance. An insignificant area of an image may be quickly excluded from close analysis, which may lead to reducing labor of reading and to achieving an effective diagnosis. Accordingly, with assistance of AI, it is possible to improve a quality of care and workflow in a medical field.

One of ordinary skill in the art may easily understand that the methods and/or processes and operations described herein may be implemented using hardware components, software components, or a combination thereof based on the example embodiments. For example, the hardware components may include a general-purpose computer and/or exclusive computing apparatus or a specific computing apparatus or a special feature or component of the specific computing apparatus. The processes may be implemented using at least one microprocessor having an internal and/or external memory, a microcontroller, an embedded microcontroller, a programmable digital signal processor or other programmable devices. In addition, or, as an alternative, the processes may be implemented using an application specific integrated circuit (ASIC), a programmable gate array, a programmable array logic (PAL), or other devices configured to process electronic signals, or combinations thereof. Targets of technical solutions of the disclosure or portions contributing to the arts may be configured in a form of program instructions performed by various computer components and stored in non-transitory computer-readable recording media. The media may include, alone or in combination with the program instructions, data files, data structures, and the like. The program instructions recorded in the media may be specially designed and configured for the example embodiments, or may be known to those skilled in the art of computer software. Examples of the media may include magnetic media such as hard disks, floppy disks, and magnetic tapes; optical media such as CD-ROM discs, DVDs, and Blu-ray; magneto-optical media such as floptical disks; and hardware devices that are specially configured to store and perform program instructions, such as ROM, RAM, flash memory, and the like. Examples of program instructions may include a machine code, such as produced by a compiler and higher language code that may be executed by a computer using an interpreter. Examples of program instructions include both machine code, such as produced by a compiler and files containing structural programming languages (such as C), object-oriented programming language (such as C++) and high or low programming languages (assembly languages, hardware technical languages, database programming languages and techniques) to run not only on one of the aforementioned devices but also a processor, a processor architecture, or a heterogeneous combination of combinations of different hardware and software components, or a machine capable of executing program instructions. Accordingly, they may include a machine language code, a byte code, and a high language code executable using an interpreter and the like.

Therefore, according to an aspect of at least one example embodiment, the aforementioned methods and combinations thereof may be implemented by one or more computing apparatuses as an executable code that performs the respective operations. According to another aspect, the methods may be implemented by systems that perform the operations and may be distributed over a plurality of devices in various manners or all of the functions may be integrated into a single exclusive, stand-alone device, or different hardware. According to another aspect, devices that perform operations associated with the aforementioned processes may include the aforementioned hardware and/or software. Such all of the sequences and combinations associated with the processes are to be included in the scope of the present disclosure.

For example, the described hardware devices may be to act as one or more software modules in order to perform the operations of the above-described example embodiments, or vice versa. The hardware devices may include a processor, such as, for example, an MPU, a CPU, a GPU, and a TPU, configured to be combined with a memory such as ROM/RAM configured to store program instructions and to execute the instructions stored in the memory, and may include a communicator capable of transmitting and receiving a signal with an external device. In addition, the hardware devices may include a keyboard, a mouse, and an external input device for receiving instructions created by developers.

While this disclosure is described with reference to specific matters such as components, some example embodiments, and drawings, they are merely provided to help general understanding of the disclosure and this disclosure is not limited to the example embodiments. It will be apparent to those skilled in the art that various alternations and modifications in forms and details may be made from the example embodiments.

Therefore, the scope of this disclosure is not defined by the example embodiments, but by the claims and their equivalents, and all variations within the scope of the claims and their equivalents are to be construed as being included in the disclosure.

Such equally or equivalently modified example embodiments may include, for example, logically equivalent methods capable of achieving the same results as those acquired by implementing the method according to the example embodiments. Accordingly, the present disclosure and the scope thereof are not limited to the aforementioned example embodiments and should be understood as a widest meaning allowable by law.

1. A method of supporting viewing of images, wherein a computing apparatus enables sequential viewing of a series of individual images in response to a specific input of an input device, and
a switching speed from a first individual image provided in a current viewing to a second individual image provided in a subsequent viewing determined according to an importance associated with at least one of the first individual image and the second individual image.

2. The method of claim 1, comprising:
acquiring, by the computing apparatus, the series of individual images;
providing, by the computing apparatus, a single image among the series of individual images as the first individual image; and
updating, by the computing apparatus, the first individual image with the second individual image determined to be provided in the subsequent viewing based on a directivity corresponding to the specific input, and the switching speed,
wherein the switching speed as determined based on at least one of an importance of the first individual image or at least one image adjacent to the first individual image, or according to a predetermined manipulation.

3. The method of claim 2,
wherein the updating the first individual image with the second individual image is performed if an input amount accumulated in response to the specific input exceeds a predetermined threshold,
wherein the second individual image is determined to be provided in the subsequent viewing based on a directivity of the input amount, and the threshold is a predetermined value having a function relationship with respect to an importance associated with at least one of the first individual image, m previous images of the first individual image, and n subsequent images of the first individual image, or designated according to a predetermined manipulation, where m and n≥1 and m and n denote a natural number.

4. A non-transitory computer-readable storage medium storing a program instructions that is executable by a computer to perform the method of claim 1.

5. A computing apparatus for supporting viewing of images, the apparatus comprising:
a communicator configured to acquire a specific input of an input device; and
a processor configured to enable sequential viewing of individual images in response to the specific input,
wherein the processor is configured to enable a switching speed from a first individual image provided in a current viewing to a second individual image provided in a subsequent viewing to be determined according to an importance associated with at least one of the first individual image and the second individual image.

6. The apparatus of claim 5,
wherein the processor is configured to provide a single image among the series of individual images as a current viewing image, and
the processor is configured to perform a process of updating the first individual image with the second individual image determined to be provided in the subsequent viewing based on a directivity corresponding to the specific input,
wherein the switching speed is determined based on at least one of an importance of the first individual image or at least one image adjacent to the first individual image, or according to a predetermined manipulation.

7. The apparatus of claim 6, wherein the process of updating is performed if an input amount accumulated in response to the specific input exceeds a predetermined threshold,
wherein the second individual image is determined to be provided in the subsequent viewing based on a directivity of the input amount, and the threshold is a predetermined value having a function relationship with respect to an importance associated with at least one of the first individual image, m previous images of the first individual image, and n subsequent images of the first individual image, or designated according to a predetermined manipulation, where m and n≥1 and m and n denote a natural number.

8. The method of claim 1, wherein the importance is determined based one of a confidence that a suspected lesion detected in an individual image is an actual lesion, a malignity of the suspected lesion and a value that is calculated based on at least of the confidence and the malignity.

9. The apparatus of claim 5, wherein the importance is determined based one of a confidence that a suspected lesion detected in an individual image is an actual lesion, a malignity of the suspected lesion and a value that is calculated based on at least of the confidence and the malignity.
